(19) United States
(12) Patent Application Publication
(10) Pub. No.: US 2015/0020811 A1
(43) Pub. Date: Jan. 22, 2015

(54) OXYGEN DELIVERY DEVICE

(71) Applicant: UPODS, LLC, Bethlehem, PA (US)

(72) Inventor: HUGH W. PAYTON, NAPLES, FL (US)

(73) Assignee: UPODS, LLC, Bethlehem, PA (US)

(21) Appl. No.: 13/944,226

(22) Filed: Jul. 17, 2013

(51) Int. Cl. A61M 16/06

(52) U.S. Cl. CPC A61M 16/0666
USPC 128/207.18

(57) ABSTRACT

An oxygen delivery device (1) having a substantially U-shaped nasal cannula (3) with a formable tip (31) that fits inside the patient's nostril and extends around to an outer surface of the patient's nostril then toward the patient's eye or ear. The substantially U-shaped cannula has a first section of formable tubing (8) then extends from the U-shaped cannula across the patient's cheek around the side of the face and over the patient's ear. A second section of formable tubing (10) extends over the ear and a coiled section of tubing then extends downward to the patient's shoulder where a clip (15) located near the coiled section of flexible tubing may be used to attach the coiled section of flexible tubing to the patient's clothing. A stabilization patch (17 or 18) further secures the tubing to the cheek and/or behind the ear of the patient.

### FIELD OF THE INVENTION

This application relates to an apparatus for supplying supplemental oxygen to a patient via a nasal orifice.

### BACKGROUND OF THE INVENTION

Dual prong nasal cannulas are generally used to deliver oxygen to a patient via the patient's nose. The most commonly used arrangement includes a dual prong nosepiece which is centered in a loop of vinyl tubing. The nosepiece openings are inserted in the nostrils with the tubing tucked behind the ears and around to the front of the patient below the chin. A slide adjustment may be used to draw it tight beneath the chin. Usually by the third or fourth day of using a dual prong nasal cannula, irritation of the skin and subcutaneous areas in contact with the cannulas and tubing begins and by the fifth day, the majority of patients have begun to use tissues and the like to relieve the soreness, pressure and irritation under the nose and around the ears. This soreness, pressure and irritation is often due to abrasion and interface pressure which is often caused by movement of the tight fitting tubing and cannulas and to the accumulation of moisture between the skin and the tubing and/or cannulas. As the slide is pulled tighter to keep the cannulas in the nostrils of the patient the irritation is only exacerbated and the subcutaneous layers of the skin are affected.

Thus, the use of a dual prong cannula can become quite uncomfortable for a patient and can lead to pressure ulcers. The comfort of the patient becomes even more critical, both to the patient and to the professionals attending the patient when the patient is also fitted with a naso-gastric or Levine tube. Now the nose becomes a fairly cluttered access route, and adhesive tape is often used, by application to the face, to get all the tubes to remain in place.

In addition to the discomfort and susceptibility to skin deterioration where supplemental oxygen is required, a patient may suffer from “free floating anxiety” as a result of reduced blood oxygen. Such a patient may believe something is wrong but cannot quite identify the problem, and may not be thinking clearly. Often such patients have feelings of claustrophobia and may attempt to remove the cannulas despite the fact that doing so may adversely affect the patient's condition. It is not uncommon to find the tubing disconnected or prongs displaced from the nose due to pulling from movement of the head, especially during sleep. Further, patient non-compliance or lack of cooperation may necessitate the use of some more expensive or aggressive means of oxygen administration, including face masks or catheters.

To an increasing extent, supplemental oxygen is used on an outpatient basis, such as in a home. Under such conditions, the cosmetics of the oxygen delivery apparatus can be important, and the commonly-used dual prong cannula can make a person feel conspicuous and, as previously mentioned, cause irritation from interface pressure.

In addition to the discomfort, skin deterioration, and cosmetic concerns posed by use of the dual prong cannulas, it has been established that single prong nasal, or unilateral, nasal catheters provide a higher inspired oxygen fraction than the dual prong cannulas.

Therefore, a need exists for an oxygen delivery device having a single prong nasal cannula that can be securely affixed to a patient while providing an adequate supply of oxygen to the patient with the least amount of irritation and/or discomfort to the patient possible.

The relevant prior art includes the following references:

| Pat. No. | | Issue/ |
|---|---|---|
| (U.S. Patent References) | Inventor | Publication Date |
| 4,660,555 | Payton | Apr. 28, 1987 |
| 4,685,456 | Smart | Aug. 11, 1987 |
| 4,736,741 | Payton et al. | Apr. 12, 1988 |
| 4,742,824 | Payton et al. | May 10, 1988 |
| 6,804,866 | Lemke et al. | Oct. 19, 2004 |
| 6,807,966 | Wright | Oct. 26, 2004 |
| WO2005/014080 | Wood | Feb. 17, 2005 |
| 6,913,017 | Roberts | Jul. 05, 2005 |
| 2008/0223375 | Cortez et al. | Sep. 18, 2008 |
| D643,113 | Butler | Aug. 09, 2011 |

### SUMMARY OF THE INVENTION

The primary object of the present invention is to provide an oxygen delivery device having a single prong nasal cannula and tubing that can be securely affixed to a patient while providing an adequate supply of oxygen to the patient with the least amount of irritation and/or discomfort to the patient possible.

The present invention fulfills the above and other objects by providing an oxygen delivery device having a single prong nasal cannula for insertion into a patient's right or left nostril to deliver oxygen to the patient. A U-shaped cannula preferably constructed of flexible or non-flexible, bendable or memory materials that fits inside the patient's nostril and extends around to an outer surface of the patient's nostril, attaches to flexible and preferably formable or bendable tubing, which tubing then extends upward toward the patient's eye or ear, across the patient's cheek around the side of the face and over the patient's ear. The tubing over the ear is also preferably made of a flexible, formable or memory material that it may be contoured over the ear and bent slightly inward against the patient's scalp behind the ear to lock the tubing in place around the ear like a temple tip of a conventional pair of eye glasses. A secondary coiled section of flexible tubing may then extend downward to the patient's shoulder. The coiled section of tubing allows the patient to move his or her head without affecting the placement of the U-shaped cannula in the nostril. Finally, a flexible piece of tubing extends directly to a supply source for supplying oxygen to the patient and preferably around the back of the patient's neck over the opposite shoulder where a clip located on the coiled tubing may be used to attach the tubing to the patient's clothing and then to the supply source for supplying oxygen.

An additional feature of the present invention is a clip with planar stabilization patch that further secures the tubing to the cheek of the patient. The clip is a semi-rigid substantially C-shaped clip located on an upper surface of the planar stabilization patch having adhesive located on a rear surface to attach the patch to the patient's cheek. After the patch is secured to the patient's cheek, the tubing may be pressed into the C-shaped clip and held in place by a pressure fit. The planar stabilization patch may also have other means of holding the tubing to the cheek such as an adhesive and so forth.

An additional feature of the present invention is a flexible stabilization patch which may be secured to the patient's cheek and/or to the skin behind the patient's ear by an adhesive. The flexible stabilization patch secures the tubing to the face and over and behind the ear by adhesive.

An additional feature of the present invention is a nasal cannula tip made of pliable or formable materials which may be affixed to the end of the cannula. The cannula tip secures the cannula within the nostril and enhances the inspired oxygen by reducing the backflow of oxygen out of the nostril.

The above and other objects, features and advantages of the present invention should become even more readily apparent to those skilled in the art upon a reading of the following detailed description in conjunction with the drawings wherein there is shown and described illustrative embodiments of the invention.

### BRIEF DESCRIPTION OF THE DRAWINGS

In the following detailed description, reference will be made to the attached drawings in which:

FIG. 1 is a front view of an oxygen delivery device of the present invention being worn by a patient;

FIG. 2 is a rear view of an oxygen delivery device of the present invention being worn by a patient;

FIG. 3 is a side view of a U-shaped cannula of the present invention constructed constructed of a non-flexible material;

FIG. 4 is a side view of a U-shaped cannula of the present invention constructed of a flexible, formable, or memory material;

FIG. 5 is a front view of a semi-rigid stabilization patch of the present invention;

FIG. 6 is a side view of a semi-rigid stabilization patch of the present invention;

FIG. 7 is an open view of a flexible stabilization patch of the present invention;

FIG. 8 is a closed view of a flexible stabilization patch of the present invention; and

FIG. 9 is a side view of a U-shaped cannula with a cannula tip of the present invention attached and constructed of pliable material.

### DESCRIPTION OF THE PREFERRED EMBODIMENTS

For purposes of describing the preferred embodiment, the terminology used in reference to the numbered accessories in the drawings is as follows:
1. oxygen delivery device, generally
2. patient
3. U-shaped nasal cannula
4. first end of U-shaped nasal cannula placed in nostril interior
5. ninety degree bend in cannula
6. second end of U-shaped nasal cannula exterior to nostril
7. juncture of cannula to first section of tubing
8. first section of tubing
9. juncture of first section of tubing to second section of tubing
10. second section of tubing
11. juncture of second section of tubing to third section of tubing
12. third section of tubing
13. juncture of third section of tubing to fourth section of tubing
14. fourth section of tubing
15. clip attaching fourth section of tubing to clothing
16. oxygen supply connector
17. semi-rigid stabilization patch
18. flexible stabilization patch
19. flexible wire
20. cannula wall
21. C-shaped clip
22. upper surface of semi-rigid stabilization patch
23. adhesive
24. rear surface of semi-rigid stabilization patch
25. open view of flexible stabilization patch
26. rear adhesive surface of flexible stabilization patch
27. tubing placement area of flexible stabilization patch
28. upper adhesive surface of flexible stabilization patch
29. closed view of flexible stabilization patch
30. outer non-adhesive surface of closed flexible stabilization patch
31. cannula tip

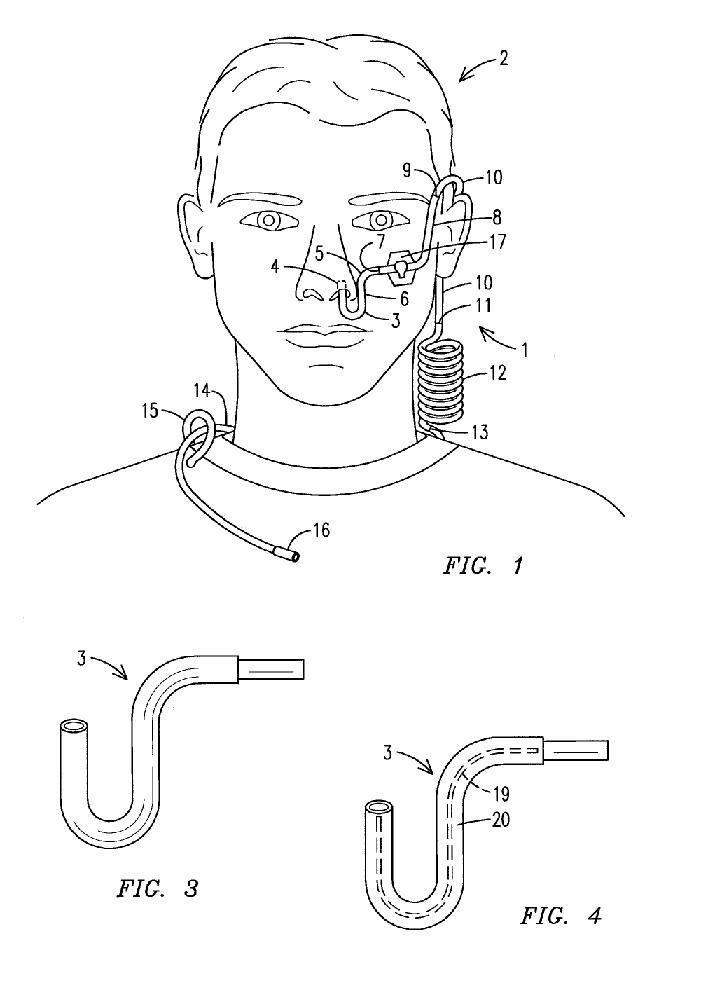

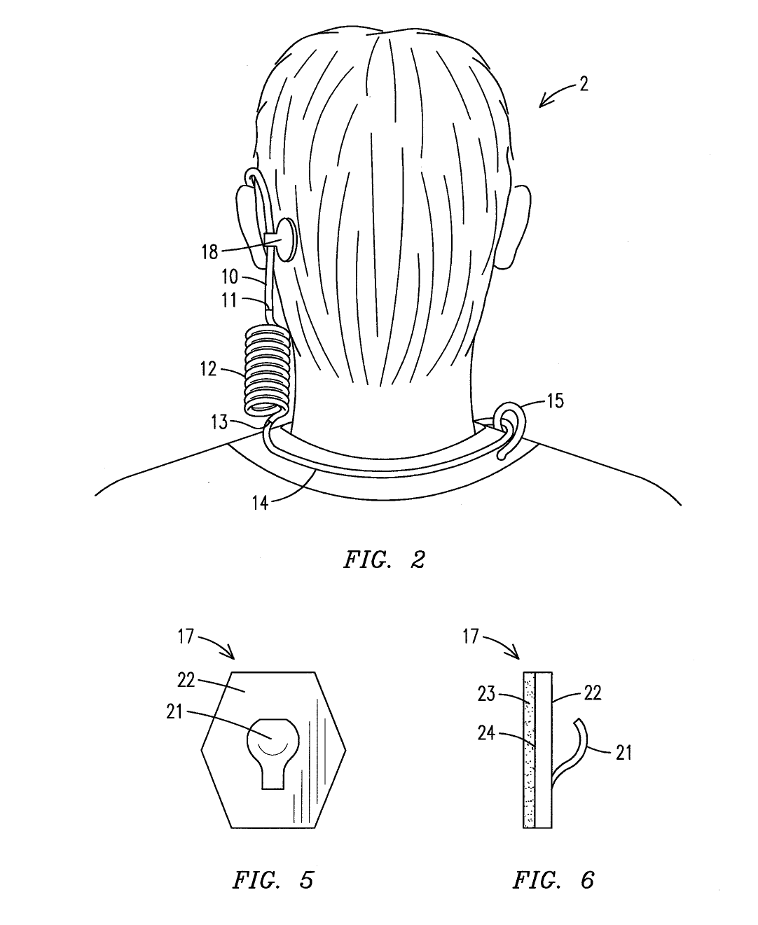

With reference to FIGS. 1 and 2, a front view and a rear view, respectively, of an oxygen delivery device 1 of the present invention being worn by a patient 2 is illustrated. The oxygen delivery device 1 comprises a substantially U-shaped nasal cannula 3 constructed from a non-flexible, flexible, formable, or memory material, or tubing having a wire embedded therein that is bendable, and which fits inside the patient's nostril and extends around to an outer surface of the patient's nostril, attaches to flexible and preferably formable or bendable tubing, which tubing then extends upward toward the patient's eye or ear. The substantially U-shaped cannula 3 has a first end 4 that fits inside the nostril from which oxygen or other gasses are delivered to the patient.

The second end 6 of the U-shaped cannula 3 may further comprise a substantially ninety degree bend 5 preferably constructed from the same material as the rest of the substantially U-shaped cannula 3 to prevent formation of kinks which could reduce or restrict the flow of oxygen and to enhance placement of the cannula and tubing on the patient's face. The substantially U-shaped cannula second end 6 is joined at the juncture 7 of cannula 3 to the first section of tubing 8. The first section of tubing 8, that is preferably flexible, formable or bendable, extends from a second end 6 of the U-shaped cannula across the patient's cheek, around the side of the face and toward the top of the patient's ear. The first section of tubing 8 may be joined at juncture 9 to a second section of tubing 10. The second section of tubing 10 extends over and behind the ear and may be flexible, formable or bendable so that it may be contoured over the ear and bent slightly inward against the patient's scalp behind the ear to secure the tubing placement. This configuration allows the tubing to be worn slightly above the ear, thereby preventing interface pressure and friction behind the ear and allowing a patient to wear eye glasses without interference from the tubing. A second section of tubing 10 is affixed to a third section of tubing 12 at a juncture 11. The third section of tubing 12 preferably consists of coiled flexible tubing which allows the patient to move his or her head without affecting the placement of the U-shaped cannula 3 in the nostril. A third section of tubing 12 is joined at juncture 13 to a fourth section of tubing 14. A fourth section of tubing 14 extends directly to the oxygen supply source and is preferably draped across the back of the patient's neck and crossing to the opposite shoulder of the nostril into which the cannula 3 is inserted and thence to the oxygen supply source. A clip 15 may be used to attach the fourth section of tubing 14 to the patient's clothing, further securing the cannula 3 placement. A fourth section of tubing 14 is attached to the oxygen supply at connector 16. Also illustrated in FIG. 1 is a semi-rigid stabilization patch 17 on the patient's face that further secures the tubing to the cheek of the patient and is discussed in greater detail below. In an alternate embodiment, the first section of tubing 8 is joined to the third section of tubing 12 at juncture 11.

With reference to FIG. 2, a rear view of an oxygen delivery device of the present invention being worn by a patient, a flexible stabilization patch 18 affixed behind the patient's ear is illustrated. This flexible stabilization patch 18 functions to additionally secure the tubing placed over the ear and reduce the movement of the tubing in the vicinity of the ear, reducing the interface pressure and friction of the tubing against the skin.

With reference to FIG. 3, a side view of a U-shaped cannula 3 of the present invention constructed out of a non-flexible tubing is illustrated, which is self explanatory when viewed in conjunction with the description above.

With reference to FIG. 4, a side view of a U-shaped cannula 3 of the present invention constructed out of a flexible, formable or memory tubing is illustrated. As illustrated here, there is a flexible wire 19 embedded in a wall 20 or proximal to the wall 20 of the U-shaped cannula 3 that retains its shape when bent or returns to its original shape after bending. However, the material used to form the flexible, formable or memory tubing may also be a semi-rigid plastic, polyurethane, elastomer or other material that retains its shape when bent without the assistance of the embedded flexible wire 19. Such tubing may also be applied to the second section of tubing 10 that fits over the ear of the patient.

With reference to FIGS. 5 and 6, a front view and a side view, respectively, of a semi-rigid stabilization patch 17 is illustrated. The semi-rigid stabilization patch 17 comprises a preferably semi-rigid substantially C-shaped clip 21 located on an upper surface 22 of a stabilization patch 17 having adhesive 23 located on a rear surface 24 to attach the stabilization patch 17 to a patient's cheek, as illustrated in FIG. 1. The first section of tubing 8 may be pressed into the C-shaped clip 21 and held in place by a pressure fit, as illustrated in FIG. 1. One or more stabilization patches 17 or 18 (as described below) may be placed on a patient's cheek or proximate to the nose to stabilize the tubing. The placement of the tubing is over areas of the face that have minimum hair growth and oil secretion, thereby ensuring the one or more stabilization patches 17 or 18 adhere to the patient's skin.

With reference to FIG. 7, an open view of a flexible stabilization patch 25, a stabilization patch which may be affixed to the face as shown in FIG. 1 in replacement of the semi-rigid stabilization patch 17 or behind the patient's ear 18 as shown in FIG. 2. The flexible stabilization patch 25 preferably should be made of flexible, breathable and non-irritating material with an adhesive side 26 which attaches to the skin and a folding adhesive section 28 which serves to secure the tubing when placed in the tubing securement area 27.

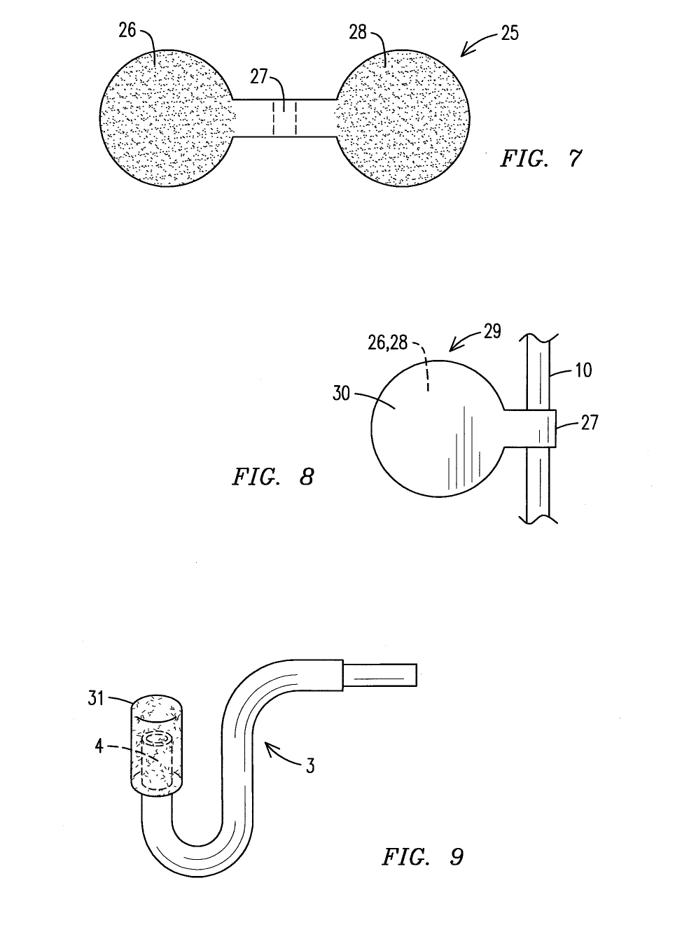

With reference to FIG. 8, a closed view of a flexible stabilization patch 29 as shown in open configuration in FIG. 7, a closed stabilization patch with tubing 10 shown stabilized in the tube securement area 29. The closed flexible stabilization patch 29 preferably should be made of the materials described for FIG. 7 with a non-adhesive upper surface 30 after closure.

With reference to FIG. 9, a side view of U-shaped cannula 3 with a cannula tip 31 affixed. The cannula tip 31 is preferably made of pliable or formable materials which may be affixed to the end of the cannula 4. The cannula tip 31 secures the cannula 4 within the nostril and enhances the inspired oxygen by reducing the backflow of oxygen out of the nostril.

It is to be understood that while a preferred embodiment of the invention is illustrated, it is not to be limited to the specific form or arrangement of parts herein described and shown. It will be apparent to those skilled in the art that various changes may be made without departing from the scope of the invention and the invention is not to be considered limited to what is shown and described in the specification and drawings.

1. An oxygen delivery device for delivering oxygen to a patient, said oxygen delivery device comprising:
a substantially U-shaped nasal cannula having a first end that is adapted to fit inside a patient's nostril and a second end adapted to extend around to an outer surface of the patient's nostril then upward toward the patient's eye; and
a first section of tubing constructed of memory material that retains its shape when bent, the first section of tubing adapted to extend from the second end of the substantially U-shaped cannula across the patient's cheek, around a side of the patient's face, and over the patient's ear; and
a second section of tubing extending from the first section of tubing to a gas supply source.

2. The oxygen delivery device of claim 1 further comprising:
a clip located proximal to the second section of tubing.

3. The oxygen delivery device of claim 1 further comprising:
a substantially ninety degree bend located between the second end of the nasal cannula and the first section of tubing.

4. (canceled)

5. The oxygen delivery device of claim 1 further comprising:
a stabilization patch having a substantially C-shaped clip on an upper surface and having an adhesive located on a rear surface to affix the stabilization patch to the patient's face and secure the first section of tubing.

6. The oxygen delivery device of claim 1 further comprising:
a stabilization patch adapted to be affixed to the patient's face and securing the first section of tubing.

7. The oxygen delivery device of claim 1 further comprising:
a stabilization patch adapted to be affixed to the skin behind the patient's ear and securing to the first section of tubing.

8. The oxygen delivery device of claim 1 further comprising:
a cannula tip attached to the first end of the substantially U-shaped cannula which is adapted to fit inside the patient's nostril.

9. An oxygen delivery device for delivering oxygen to a patient, said oxygen delivery device comprising:
a substantially U-shaped nasal cannula having a first end that is adapted to fit inside a patient's nostril and a second end adapted to extend around to an outer surface of the patient's nostril then upward toward the patient's eye; and
a first section of tubing constructed of memory material that retains its shape when bent, the first section of tubing adapted to extend from the second end of the substantially U-shaped cannula across the patient's cheek, around a side of the patient's face, and toward the patient's ear; and
a second section of tubing adapted to extend from the first section of tubing over the patient's ear; and
a third section of tubing extending from the second section of tubing to a gas supply source.

10. The oxygen delivery device of claim 9 further comprising:
a clip located proximal to the third section of tubing.

11. The oxygen delivery device of claim 9 further comprising:
a substantially ninety degree bend located between the second end of the nasal cannula and the first section of tubing.

12. (canceled)

13. The oxygen delivery device of claim 9 further comprising:
a stabilization patch having a substantially C-shaped clip on an upper surface and having an adhesive located on a rear surface to affix the stabilization patch to the patient's face and secure the first section of tubing.

14. The oxygen delivery device of claim 9 further comprising:
a stabilization patch adapted to be affixed to the patient's face and securing the first section of tubing.

15. The oxygen delivery device of claim 9 further comprising:
a stabilization patch adapted to be affixed to the skin behind the patient's ear and securing the second section of tubing.

16. The oxygen delivery device of claim 9 further comprising:
a cannula tip attached to the first end of the substantially U-shaped cannula which is adapted to fit inside the patient's nostril.

17. An oxygen delivery device for delivering oxygen to a patient, said oxygen delivery device comprising:
a substantially U-shaped nasal cannula having a first end that is adapted to fit inside a patient's nostril and a second end adapted to extend around to an outer surface of the patient's nostril then upward toward the patient's eye; and
a first section of tubing constructed of memory material that retains its shape when bent, the first section of tubing adapted to extend from the second end of the substantially U-shaped cannula across the patient's cheek, around a side of the patient's face, and toward the patient's ear; and
a second section of tubing adapted to extend from the first section of tubing over the patient's ear; and
a third section of coiled tubing adapted to extend from the second section of tubing to a fourth section of tubing; and
the fourth section of tubing extending from the third section of tubing to a gas supply source.

18. The oxygen delivery device of claim 17 further comprising:
a clip located proximal to the fourth section of tubing.

19. The oxygen delivery device of claim 17 further comprising:
a substantially ninety degree bend located between the second end of the nasal cannula and the first section of tubing.

20. (canceled)

21. The oxygen delivery device of claim 17 further comprising:
a stabilization patch having a substantially C-shaped clip on an upper surface and having an adhesive located on a rear surface to affix the stabilization patch to the patient's face and secure the first section of tubing.

22. The oxygen delivery device of claim 17 further comprising:
a stabilization patch adapted to be affixed to the patient's face and securing the first section of tubing.

23. The oxygen delivery device of claim 17 further comprising:
a stabilization patch adapted to be affixed to the skin behind the patient's ear and securing the second section of tubing.

24. The oxygen delivery device of claim 17 further comprising:
a cannula tip attached to the first end of the substantially U-shaped cannula which is adapted to fit inside the patient's nostril.

25. An oxygen delivery device for delivering oxygen to a patient, said oxygen delivery device comprising:
a substantially U-shaped nasal cannula having a first end that is adapted to fit inside a patient's nostril and a second end adapted to extend around to an outer surface of the patient's nostril then upward toward the patient's eye; and
a first section of tubing constructed of memory material that retains its shape when bent, the first section of tubing adapted to extend from the second end of the substantially U-shaped cannula across the patient's cheek, around a side of the patient's face, and over the patient's ear; and
a second section of tubing extending from the first section of tubing to a gas supply source; and
a stabilization patch adapted to be affixed to the patient's face and securing the first section of tubing; and
a stabilization patch adapted to be affixed to the skin behind the patient's ear and securing the first section of tubing.

26. The oxygen delivery device of claim 25 further comprising:
a clip located proximal to the second section of tubing.

27. The oxygen delivery device of claim 25 further comprising:
a substantially ninety degree bend located between the second end of the nasal cannula and the first section of tubing.

28. (canceled)

29. The oxygen delivery device of claim 25 further comprising:
the stabilization patch having a substantially C-shaped clip on an upper surface and having an adhesive located on a rear surface to affix the stabilization patch to the patient's face and secure the first section of tubing.

30. The oxygen delivery device of claim 25 further comprising:
a cannula tip attached to the first end of the substantially U-shaped cannula which is adapted to fit inside the patient's nostril.

31. The oxygen delivery device of claim 1, wherein the substantially U-shaped cannula is constructed of memory material that retains its shape when bent.

32. The oxygen delivery device of claim 1, wherein the substantially U-shaped cannula comprises a tubing having a wire embedded therein that is bendable.

33. The oxygen delivery device of claim 1, wherein the substantially U-shaped cannula is constructed of non-flexible material.

34. The oxygen delivery device of claim 1, wherein the substantially U-shaped cannula is constructed of flexible material.
